(19) United States
(12) Patent Application Publication
(10) Pub. No.: US 2025/0123229 A1
(43) Pub. Date: Apr. 17, 2025

(54) LIQUID SENSOR AND DETECTION METHOD

(71) Applicant: PILLAR Corporation, Osaka (JP)

(72) Inventors: Kentaro WATANABE, Osaka (JP); Akira NAKATSU, Osaka (JP); Yusuke NATSUHARA, Osaka (JP)

(21) Appl. No.: 18/915,664

(22) Filed: Oct. 15, 2024

(30) Foreign Application Priority Data
Oct. 17, 2023 (JP) 2023-178810
Feb. 15, 2024 (JP) 2024-021143

(51) Int. Cl. G01N 27/06; G01N 27/04; G01N 27/22; G01N 33/28

(52) U.S. Cl. CPC G01N 27/06; G01N 27/046; G01N 27/22; G01N 33/2888

(57) ABSTRACT

A liquid sensor and a detection method detect a parameter correlating with a degradation level of a liquid. The liquid sensor includes a pair of electrodes immersed in the liquid and a control unit. The control unit sweeps a frequency of an AC voltage applied between the pair of electrodes and detects an impedance of the liquid for each frequency and calculates a second parameter relating to a first parameter correlating with the impedance or the impedance itself. The second parameter is a difference between a first degree of change and a second degree of change. The first degree of change and the second degree of change each indicates an amount of change in the first parameter relative to an amount of change in the frequency. The control unit further detects a third parameter correlating with a degradation level of the liquid based on the second parameter.

### TECHNICAL FIELD

The present invention relates to a liquid sensor and a detection method.

### BACKGROUND ART

Japanese Patent No. 6910037 (Patent Document 1) discloses an oil condition determination system. In this oil condition determination system, the resistance value of an oil is measured, and if a tendency of change in the resistance value of the oil has changed from a decreasing tendency to an increasing tendency, it is determined that an oxidation condition of the oil has changed (see Patent

Document 1).
Patent Document 1: Japanese Patent No. 6910037

### SUMMARY OF THE INVENTION

In the oil condition determination system disclosed in Patent Document 1 described above, changes in the oxidation condition of the oil (an example of a “liquid”) are determined, but a parameter correlating with a degradation level of the oil is not detected.

The present invention was made to solve the above problem and has an object of providing a liquid sensor and a detection method that make it possible to detect a parameter correlating with a degradation level of a liquid.

A liquid sensor according to an aspect of the present invention includes a pair of electrodes and a control unit. The pair of electrodes are used while being immersed in a liquid. The control unit sweeps a frequency of an AC voltage applied between the pair of electrodes. The control unit detects an impedance of the liquid for each frequency and calculates a second parameter relating to a first parameter that is a value correlating with the impedance or the impedance itself. The second parameter is a difference between a first degree of change and a second degree of change. The first degree of change and the second degree of change each indicate an amount of change in the first parameter relative to an amount of change in the frequency. The liquid sensor further includes a storage unit. Relationship information indicating a correspondence between the second parameter and a third parameter correlating with a degradation level of the liquid is stored in the storage unit. The control unit detects the third parameter based on the second parameter and the relationship information.

The inventor(s) of the present invention found that there is a relatively high correlation between the second parameter and the third parameter correlating with the degradation level of the liquid. In this liquid sensor, the third parameter is detected based on the relationship information indicating the correspondence between the second parameter and the third parameter and the calculated second parameter. Therefore, it is possible to detect the third parameter relatively precisely with use of the liquid sensor.

In the liquid sensor, the first parameter may be a resistance value of the liquid or a capacitance of the liquid.

In the liquid sensor, the third parameter may be a base number of the liquid or an acid value of the liquid.

The liquid sensor may further include a temperature sensor configured to detect a temperature of the liquid, the relationship information may be set for each temperature of the liquid, and the control unit may detect the third parameter based on the temperature of the liquid, the relationship information corresponding to the temperature of the liquid, and the second parameter.

The inventor(s) of the present invention found that the relationship between the third parameter and the second parameter may be affected by the temperature of the liquid. In this liquid sensor, the third parameter is detected based on the temperature of the liquid, the relationship information corresponding to the temperature of the liquid, and the second parameter. Accordingly, the third parameter is detected with consideration given to the temperature of the liquid, and therefore, it is possible to detect the third parameter more precisely with use of this liquid sensor.

In the liquid sensor, the control unit may switch the first parameter to the resistance value or the capacitance according to a detected value of the third parameter.

The inventor(s) found that either of a second parameter calculated based on the resistance value and a second parameter calculated based on the capacitance has a higher degree of correlation with the third parameter varies according to the value of the third parameter. In this liquid sensor, the first parameter is switched to the resistance value or the capacitance according to the detected value of the third parameter. Accordingly, a second parameter that has a higher degree of correlation with the third parameter is used to detect the third parameter, and therefore, it is possible to detect the third parameter more precisely with use of this liquid sensor.

A detection method according to another aspect of the present invention includes: a step of sweeping a frequency of an AC voltage applied between a pair of electrodes immersed in a liquid; a step of detecting an impedance of the liquid for each frequency; and a step of calculating a second parameter relating to a first parameter that is a value correlating with the impedance or the impedance itself. The second parameter is a difference between a first degree of change and a second degree of change. The first degree of change and the second degree of change each indicate an amount of change in the first parameter relative to an amount of change in the frequency. The detection method further includes: a step of storing relationship information indicating a correspondence between the second parameter and a third parameter correlating with a degradation level of the liquid: and a step of detecting the third parameter based on the second parameter and the relationship information.

In this detection method, the third parameter is detected based on the relationship information indicating the correspondence between the second parameter and the third parameter and the calculated second parameter. Therefore, it is possible to detect the third parameter relatively precisely with use of this detection method.

According to the present invention, it is possible to provide a liquid sensor and a detection method that make it possible to detect a parameter correlating with a degradation level of a liquid.

### BRIEF DESCRIPTION OF THE DRAWINGS

FIG. 1 is a diagram schematically showing a configuration of a liquid sensor.

FIG. 2 is a diagram schematically showing a parallel equivalent circuit of a liquid.

FIG. 3 is a diagram schematically showing an example of a relationship between an AC voltage (input signal) applied between a pair of electrodes and a current (measurement signal) generated in a circuit including the pair of electrodes.

FIG. 4 is a diagram schematically showing a relationship between an impedance, a resistance value, a capacitance, and a phase difference.

FIG. 5 is a flowchart showing an example of a relationship information generation procedure.

FIG. 6 is a diagram schematically showing an example of a graph generated by detecting a resistance value.

FIG. 7 is a diagram showing a method for calculating a first degree of change and a second degree of change.

FIG. 8 is a diagram showing an example of a relationship between a base number (estimated value) of a liquid detected with use of relationship information and an analysis value of the base number of the liquid.

FIG. 9 is a diagram showing an example of a relationship between a base number (estimated value) of a liquid detected with use of relationship information when a single degree of change is used as a second parameter and an analysis value of the base number of the liquid.

FIG. 10 is a flowchart showing a procedure for detecting the base number of a liquid with use of the liquid sensor.

FIG. 11 is a diagram showing an example of a relationship between an acid value (estimated value) of a liquid detected with use of relationship information when the acid value of the liquid is a third parameter and an analysis value of the acid value of the liquid.

FIG. 12 is a flowchart showing a procedure for specifying relationship information to be used to detect the third parameter.

FIG. 13 is a diagram for describing change of a first parameter.

FIG. 14 is a flowchart showing another example of the procedure for detecting the base number of the liquid with use of the liquid sensor.

### EMBODIMENTS OF THE INVENTION

The following describes an embodiment according to an aspect of the present invention (hereinafter also referred to as “the present embodiment”) in detail with reference to the drawings. Note that the same or corresponding elements in the drawings are denoted by the same reference numerals, and redundant descriptions thereof are omitted. Also, the drawings are schematic drawings in which some elements are omitted or exaggerated as appropriate to facilitate understanding.

### 1. Configuration of Liquid Sensor

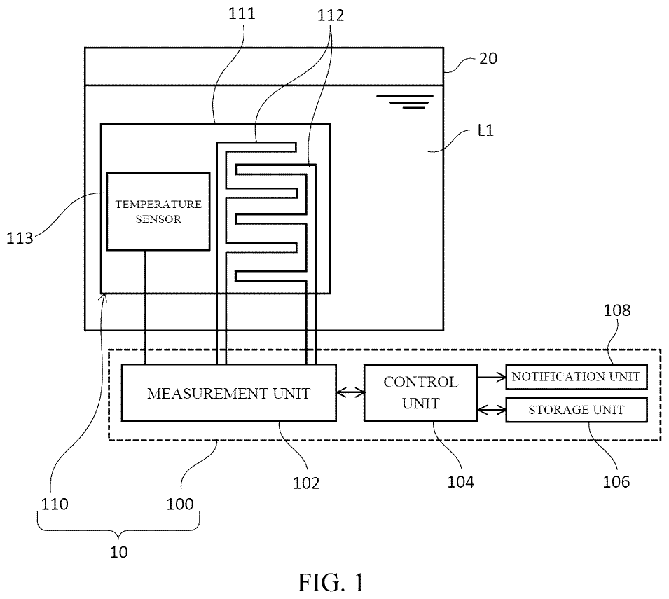

FIG. 1 is a diagram schematically showing a configuration of a liquid sensor 10 according to the present embodiment. The liquid sensor 10 is attached to the inside of a tank 20 and configured to detect a parameter (e.g., the base number) correlating with a degradation level of a liquid L1 (e.g., an oil). For example, the liquid sensor 10 is attached to the inside of an oil tank or a pipe included in a vehicle or the like and detects the base number of an oil. The base number of an oil correlates with a degradation level of the oil. The liquid sensor 10 is used while at least a portion thereof is immersed in the liquid L1.

As shown in FIG. 1, the liquid sensor 10 includes a detection system 100 and a substrate 110. The substrate 110 is immersed in the liquid L1 when the liquid sensor 10 is used. The substrate 110 has a substantially rectangular shape in a plan view, for example. The substrate 110 includes a substrate body 111, a pair of electrodes 112, and a temperature sensor 113. The substrate body 111 is a so-called fluorocarbon resin substrate. The fluorocarbon resin substrate has excellent weather resistance and chemical resistance, and therefore, the substrate 110 including the substrate body 111 can withstand use in a severe environment. Note that the substrate body 111 does not necessarily have to be constituted by a fluorocarbon resin substrate, but is preferably constituted by a substrate that has excellent chemical resistance, for example.

The pair of electrodes 112 are formed on the substrate body 111. The pair of electrodes 112 are used to detect the impedance of the liquid L1. Each electrode 112 has a comb teeth shape. The pair of electrodes 112 are arranged on the substrate 110 such that teeth portions of the respective electrodes 112 are alternately arranged. The pair of electrodes 112 are formed by performing patterning on a conductive layer formed on a surface of the substrate body 111, for example.

The temperature sensor 113 is mounted on the substrate body 111. The temperature sensor 113 is used to detect the temperature of the liquid L1. The temperature sensor 113 is constituted by a temperature detecting element such as a resistance temperature detector (RTD), a thermistor, or a thermocouple, for example. Note that the temperature sensor 113 does not necessarily have to be provided.

The detection system 100 includes a storage unit 106, a measurement unit 102, a control unit 104, and a notification unit 108. The storage unit 106 is constituted by an auxiliary storage device such as a flash memory, for example. “Relationship information” that is used to detect the base number of the liquid L1 is stored in the storage unit 106, for example. The relationship information will be described later in detail.

The measurement unit 102 includes a power source and an ammeter, for example. The measurement unit 102 applies a measurement voltage (AC voltage) between the pair of electrodes 112 and measures a current generated in a circuit including the pair of electrodes 112. The measurement unit 102 also applies a measurement voltage to the temperature sensor 113 and measures a current generated in a circuit including the temperature sensor 113, for example.

The control unit 104 includes an operation circuit, a CPU (Central Processing Unit), a RAM (Random Access Memory), and a ROM (Read Only Memory), for example. The control unit 104 detects an impedance between the pair of electrodes 112 based on the value of the voltage applied between the pair of electrodes 112 and the value of the current generated in the circuit including the pair of electrodes 112, for example. When entireties of the pair of electrodes 112 are immersed in the liquid L1, the impedance between the pair of electrodes 112 is taken to be the impedance of the liquid L1.

Although details will be described later, the control unit 104 uses an electrical resistance value (hereinafter also simply referred to as “the resistance value”) of the liquid L1 to detect the base number of the liquid L1. The control unit 104 calculates the resistance value of the liquid L1 based on the impedance of the liquid L1. The following describes an example of a procedure for calculating the resistance value of the liquid L1 based on the impedance of the liquid L1.

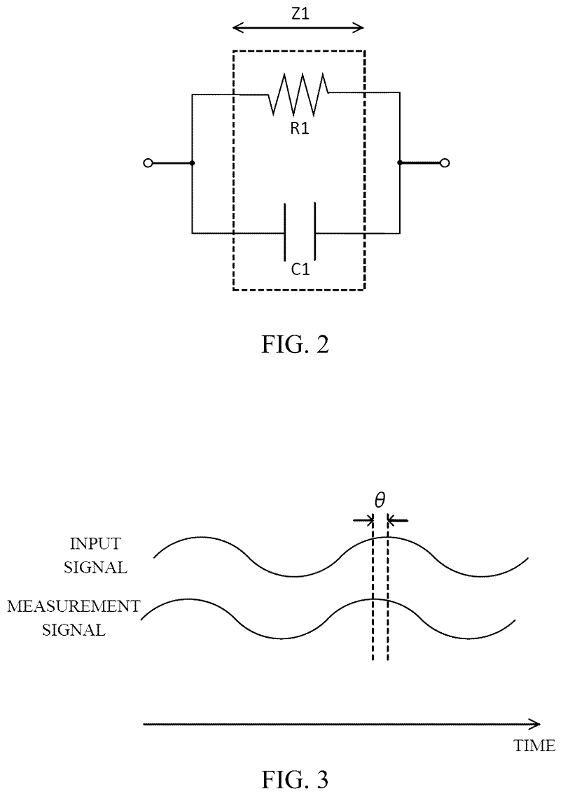

FIG. 2 is a diagram schematically showing a parallel equivalent circuit of the liquid L1. As shown in FIG. 2, the liquid L1 can be considered to be electrically equivalent to a circuit in which a resistor and a capacitor are connected in parallel. That is to say, an impedance Z1 of the liquid L1 can be divided into a resistance value R1 and a capacitance C1.

FIG. 3 is a diagram schematically showing an example of a relationship between an AC voltage (input signal) applied between the pair of electrodes 112 and a current (measurement signal) generated in the circuit including the pair of electrodes 112. In FIG. 3, the horizontal axis indicates time, and the vertical axis indicates a voltage value or a current value. In this example, there is a phase difference θ between the input signal and the measurement signal. The control unit 104 detects the phase difference θ based on the input signal and the measurement signal.

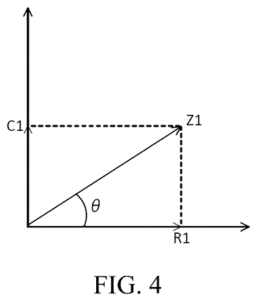

FIG. 4 is a diagram schematically showing a relationship between the impedance Z1, the resistance value R1, the capacitance C1, and the phase difference θ. As shown in FIG. 4, the control unit 104 calculates the resistance value R1 by multiplying a detected impedance Z1 by cos θ. The control unit 104 calculates the resistance value R1 of the liquid L1 based on the impedance Z1 of the liquid L1 as described above. Although details will be described later, the control unit 104 detects the base number of the liquid L1 by using the resistance value R1 of the liquid L1 and the relationship information, for example.

Referring back to FIG. 1, the control unit 104 detects a resistance value of the temperature sensor 113 based on a voltage applied to the temperature sensor 113 and a current generated in a circuit including the temperature sensor 113, for example. The control unit 104 detects the temperature of the liquid L1 based on the resistance value of the temperature sensor 113. A relationship between the temperature and the resistance value is stored in the storage unit 106 in advance, for example.

The notification unit 108 includes a display, for example. The notification unit 108 displays an image indicating a base number detected by the control unit 104, for example. Thus, a user is notified of the base number of the liquid L1.

### 2. Detection of Base Number Using Relationship Information

There is a technique for determining a timing for replacing an oil (an example of the liquid L1) by detecting a change in an oxidation condition of the oil, for example. However, a parameter (e.g., the base number) correlating with a degradation level of the liquid L1 is not directly detected with such a technique. On the other hand, if a parameter correlating with the degradation level of the liquid L1 is directly detected, it is possible to more precisely determine the timing for replacing the liquid L1, for example.

A first parameter, a second parameter, and a third parameter are defined as follows. The first parameter is a parameter (e.g., the resistance value R1 of the liquid L1) corresponding to a value correlating with the impedance Z1 of the liquid L1 or the impedance Z1 itself. The second parameter is a difference between a first degree of change and a second degree of change. The control unit 104 is configured to sweep the frequency of the measurement voltage applied between the pair of electrodes 112. The control unit 104 is configured to detect a resistance value R1 of the liquid L1 when the measurement voltage is applied at each of a plurality of predetermined frequencies (e.g., frequencies f1, f2, f3, and f4 (f1<f2<f3<f4)). The first degree of change and the second degree of change each indicate an amount of change in the first parameter relative to an amount of change in the frequency (e.g., the first degree of change is an amount of change in the first parameter relative to a difference (f4−f1) between the frequency f4 and the frequency f1, and the second degree of change is an amount of change in the first parameter relative to a difference (f3−f2) between the frequency f3 and the frequency f2). The third parameter is a parameter (e.g., the base number of the liquid L1) correlating with the degradation level of the liquid L1.

The inventor(s) of the present invention found that there is a relatively high correlation between the second parameter and the third parameter correlating with the degradation level of the liquid L1. In the liquid sensor 10 according to the present embodiment, the relationship information described above indicates the correspondence between the third parameter and the second parameter, and the third parameter is detected based on the calculated second parameter and the relationship information. Therefore, it is possible to detect the third parameter relatively precisely with use of the liquid sensor 10. As described above, the relationship information is generated in advance and stored in the storage unit 106. The following describes a relationship information generation procedure.

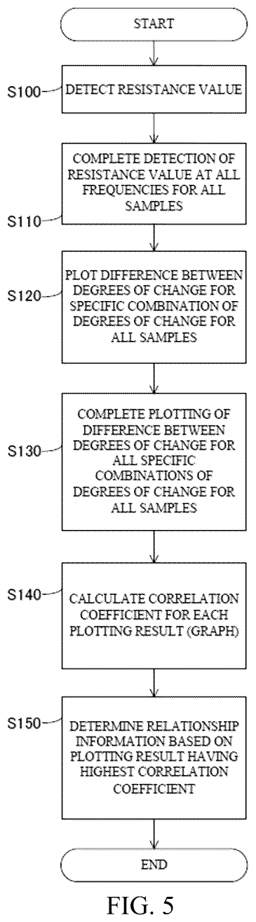

FIG. 5 is a flowchart showing an example of the relationship information generation procedure. Each step shown in this flowchart is performed by the control unit 104 or a worker. Note that a plurality of samples (liquids L1) having mutually different base numbers are prepared in advance, and the procedure shown in this flowchart is started in a state where any of the plurality of samples is placed in the tank 20 (FIG. 1). Also, the actual base numbers of the plurality of samples are measured in advance.

As shown in FIG. 5, the control unit 104 detects the resistance value R1 of a liquid L1 while a measurement voltage is applied between the pair of electrodes 112 at each frequency (step S100) and completes the detection of the resistance value at all frequencies determined in advance for all of the samples (step S110).

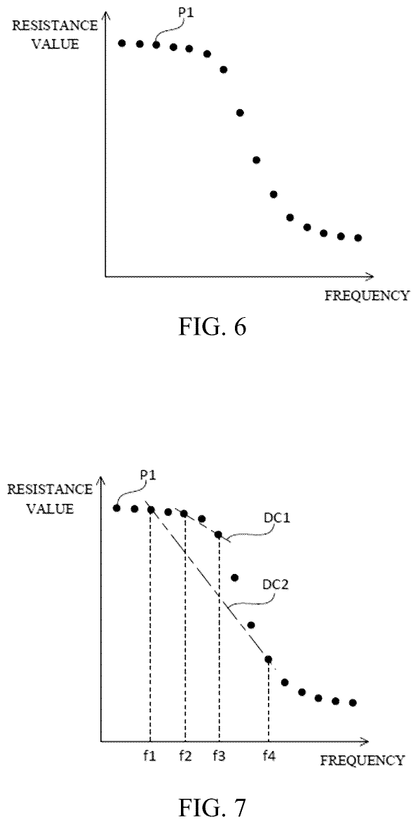

FIG. 6 is a diagram schematically showing an example of a graph generated by detecting the resistance value R1 of the liquid L1. This diagram shows a relationship between a frequency of the measurement voltage and the resistance value R1 of a specific sample. In FIG. 6, the horizontal axis indicates the frequency of the measurement voltage, and the vertical axis indicates the detected resistance value R1. Each point P1 indicates the resistance value R1 of the specific sample (liquid L1) when the measurement voltage with a specific frequency is applied between the pair of electrodes 112. Graphs like that shown in FIG. 6 are generated for all of the samples by repeatedly performing sweeping of the measurement voltage and detection of the resistance value R1 of each sample.

FIG. 7 is a diagram for explaining the first degree of change and the second degree of change. In FIG. 7, each point P1 indicates the resistance value R1 of the specific sample (liquid L1) when the measurement voltage with a specific frequency is applied between the pair of electrodes 112 as described above. A degree DC1 of change is an example of the first degree of change, and a degree DC2 of change is an example of the second degree of change. Each of the degrees DC1 and DC2 of change indicates an amount of change in the resistance value R1 (first parameter) relative to an amount of change in the frequency of the measurement voltage. In this example, the degree DC1 of change indicates an amount of change in the resistance value R1 relative to a difference (f3−f2) between the frequency f3 and the frequency f2, and the degree DC2 of change indicates an amount of change in the resistance value R1 relative to a difference (f4−f1) between the frequency f4 and the frequency f1. Each of the degrees DC1 and DC2 of change can also be said to be an inclination of a straight line connecting two points each indicating a correspondence between a frequency of the measurement voltage and a resistance value R1.

Referring back to FIG. 5, the worker plots a difference between the first degree of change and the second degree of change for a specific combination of the first and second degrees of change, with respect to all of the samples (step S120). Thus, a graph showing a correspondence between the difference between the first and second degrees of change and the base number of the liquid L1 is generated. The worker completes plotting the difference between the first degree of change and the second degree of change for all specific combinations of the first and second degrees of change, with respect to all of the samples (step S130). Thus, graphs showing the correspondence between the difference between the first and second degrees of change and the base number of the liquid L1 are generated for the respective combinations of the first and second degrees of change.

The worker calculates a correlation coefficient based on each of the generated graphs (plotting results) (step S140). Various known methods are used to calculate the correlation coefficient, for example. The correlation coefficient is calculated by using a computer, for example. The worker generates “relationship information” based on a graph for which the correlation coefficient is the highest (step S150). The relationship information is generated through this procedure. The relationship information may be a linear function, a quadratic function, or a cubic or higher degree function, for example. Note that the relationship information indicates a correspondence between the difference between the first and second degrees of change and the base number (an example of the third parameter) of the liquid L1, for a combination of the first and second degrees of change used to generate the graph for which the correlation coefficient is the highest. That is to say, two frequencies used to calculate the first degree of change and two frequencies used to calculate the second degree of change are determined in advance in the relationship information.

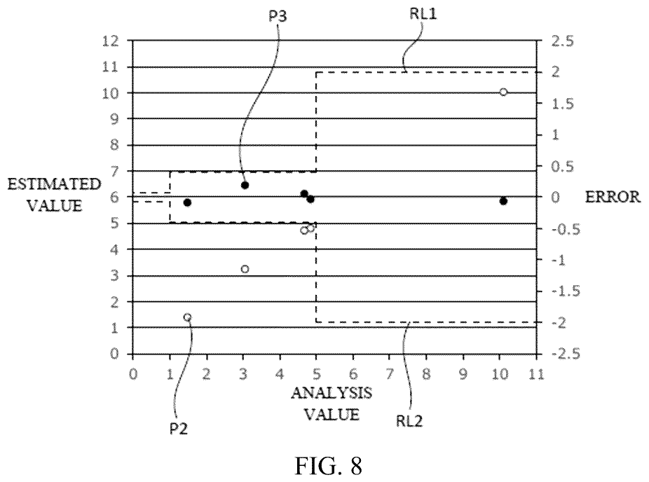

FIG. 8 is a diagram showing an example of a relationship between a base number (estimated value) of a liquid L1 detected by the liquid sensor 10 according to the present embodiment with use of the relationship information and an analysis value of the base number of the liquid L1. In FIG. 8, the horizontal axis indicates the analysis value, and the vertical axis indicates the estimated value or an error. Each white point P2 indicates an estimated value regarding the liquid L1 that has a specific analysis value, and each black point P3 indicates an error between the estimated value and the analysis value. A reference line RL1 shows an upper limit of the error specified in the JIS standards (JIS K 2501:2003 potentiometric titration method (base number/hydrochloric acid method)), and a reference line RL2 shows a lower limit of the error specified in the JIS standards.

As shown in FIG. 8, errors of respective estimated values fall within the range of errors specified in the JIS standards. This indicates that the second parameter (the difference between the first degree of change and the second degree of change) has a high correlation with the third parameter (e.g., the base number). In the liquid sensor 10 according to the present embodiment, the relationship information indicates the correspondence between the third parameter and the second parameter, and the third parameter is detected based on the calculated second parameter and the relationship information. Therefore, it is possible to detect the third parameter relatively precisely with use of the liquid sensor 10.

Assume that the second parameter is not a difference between the first degree of change and the second degree of change but is a single degree of change (an amount of change in the first parameter relative to an amount of change in the frequency). Assume that, in this case, relationship information indicating a correspondence between the third parameter and the second parameter is generated in advance. Also, the base number of the liquid L1 is estimated with use of the relationship information.

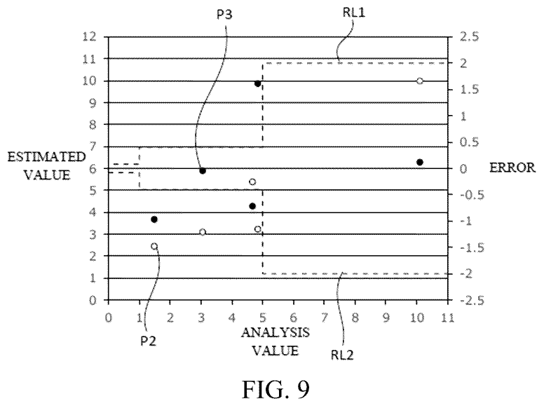

FIG. 9 is a diagram showing an example of a relationship between a base number (estimated value) of the liquid L1 detected with use of the relationship information when a single degree of change is used as the second parameter and an analysis value of the base number of the liquid L1. As shown in FIG. 9, errors of some estimated values do not fall within the range of errors specified in the JIS standards. It can be found that, when a single degree of change is used as the second parameter, the second parameter does not have a high correlation with the third parameter.

In the liquid sensor 10 according to the present embodiment, a difference between the first degree of change and the second degree of change is used as the second parameter, and the third parameter is detected based on the calculated second parameter and the relationship information. Therefore, it is possible to detect the third parameter relatively precisely with use of the liquid sensor 10.

### 3. Base Number Detection Procedure

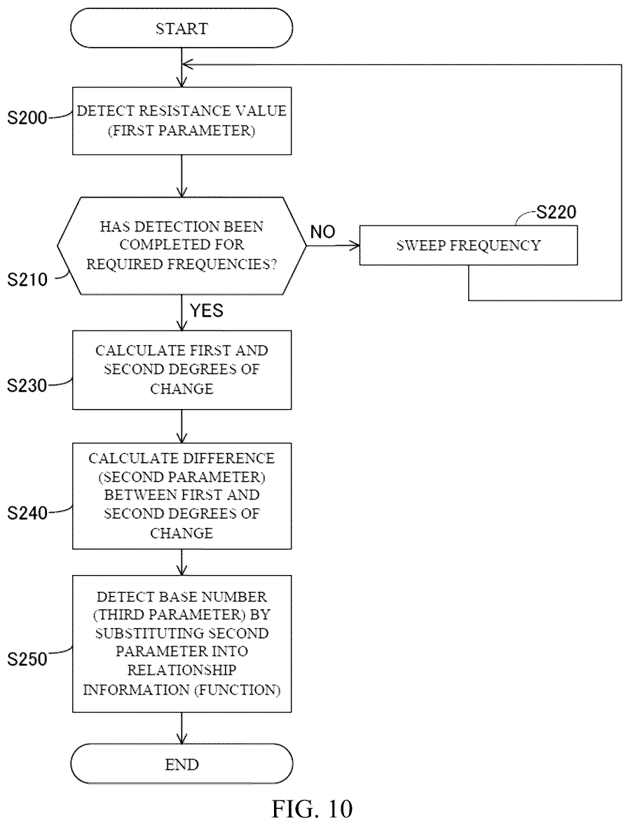

FIG. 10 is a flowchart showing a procedure for detecting the base number of the liquid L1 with use of the liquid sensor 10. Processing shown in this flowchart is executed by the control unit 104

As shown in FIG. 10, the control unit 104 detects the resistance value R1 (first parameter) of the liquid L1 while a measurement voltage is applied between the pair of electrodes 112 at a predetermined frequency (e.g., any of four frequencies determined in advance in the relationship information) (step S200). The control unit 104 determines whether or not the resistance value R1 has been detected while the measurement voltage is applied between the pair of electrodes 112 at each of a plurality of (e.g., four) predetermined frequencies determined in advance (step S210). Upon determining that the resistance value R1 has not been detected with respect to at least one frequency (NO in step S210), the control unit 104 controls the measurement unit 102 to sweep the frequency of the measurement voltage (step S220). Thereafter, the processing performed in step S200 is performed again.

Upon determining that the resistance value R1 has been detected with respect to all of the predetermined frequencies (YES in step S210), the control unit 104 calculates the first and second degrees of change based on detection results of the resistance value R1 (step S230). The control unit 104 calculates a difference (second parameter) between the first degree of change and the second degree of change (step S240). The control unit 104 detects the base number (third parameter) of the liquid L1 by substituting the second parameter into the relationship information (e.g., a function) stored in the storage unit 106 (step S250).

### 4. Features

As described above, the liquid sensor 10 according to the present embodiment includes the pair of electrodes 112 and the control unit 104. The pair of electrodes 112 are used while being immersed in a liquid L1. The control unit 104 sweeps the frequency of an AC voltage applied between the pair of electrodes 112. The control unit 104 detects an impedance of the liquid L1 for each frequency and calculates a second parameter relating to a first parameter (e.g., the resistance value R1) that is a value correlating with the impedance or the impedance itself. The second parameter is a difference between a first degree of change and a second degree of change. The first degree of change and the second degree of change each indicate an amount of change in the first parameter relative to an amount of change in the frequency. The liquid sensor 10 further includes the storage unit 106. Relationship information indicating a correspondence between the second parameter and a third parameter (e.g., the base number) correlating with a degradation level of the liquid L1 is stored in the storage unit 106. The control unit 104 detects the third parameter based on the second parameter and the relationship information. The second parameter has a relatively high correlation with the third parameter, and the third parameter is detected based on the second parameter and the relationship information indicating the correspondence between the third parameter and the second parameter, and therefore, it is possible to detect the third parameter relatively precisely with use of the liquid sensor 10.

### 5. Other Embodiments

The idea of the above embodiment is not limited to the embodiment described above. The following describes an example of other embodiments to which the idea of the above embodiment is applicable.

<5-1>

In the above embodiment, the first parameter is the resistance value R1 of the liquid L1. However, the first parameter does not necessarily have to be the resistance value R1 of the liquid L1. The first parameter may also be the capacitance C1 of the liquid L1 or the impedance Z1 of the liquid L1, for example. Relationship information may also be generated in advance with use of the capacitance C1 or the impedance Z1 of the liquid L1 as the first parameter, and the third parameter of the liquid L1 may also be detected in accordance with the flowchart shown in FIG. 10 with use of the capacitance C1 or the impedance Z1 of the liquid L1 as the first parameter.

<5-2>

In the above embodiment, the third parameter is the base number of the liquid L1. However, the third parameter does not necessarily have to be the base number of the liquid L1. The third parameter may also be the acid value of the liquid L1, for example. Relationship information may also be generated in advance with use of the acid value of the liquid L1 as the third parameter, and the third parameter of the liquid L1 may also be detected in accordance with the flowchart shown in FIG. 10 with use of the acid value of the liquid L1 as the third parameter.

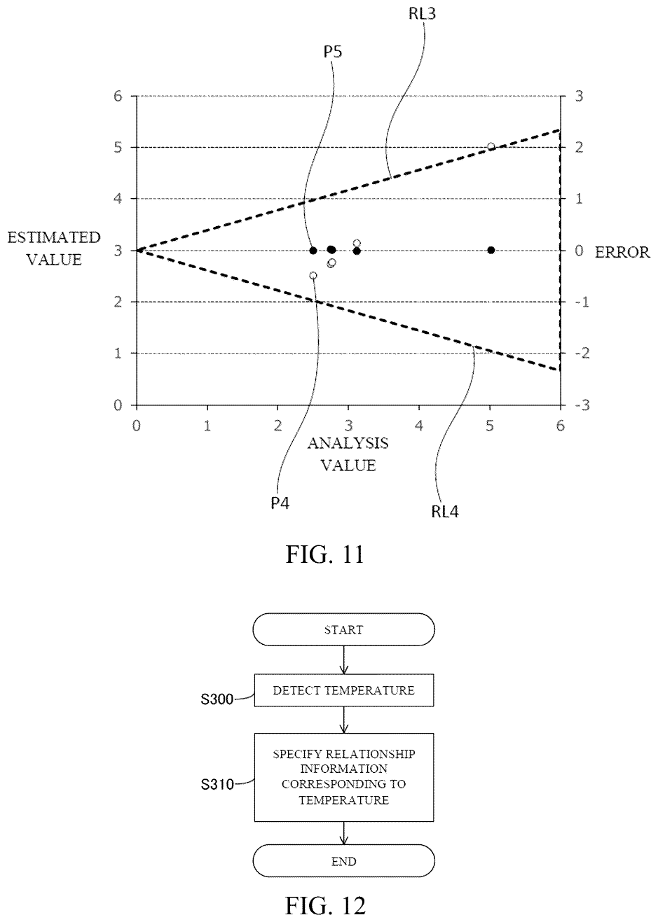

FIG. 11 is a diagram showing an example of a relationship between an acid value (estimated value) of the liquid L1 detected with use of relationship information when the acid value of the liquid L1 is the third parameter and an analysis value of the acid value of the liquid L1. In FIG. 11, the horizontal axis indicates the analysis value, and the vertical axis indicates the estimated value or an error. Each white point P4 indicates an estimated value (acid vale) regarding the liquid L1 that has a specific analysis value (acid value), and each black point P5 indicates an error between the estimated value and the analysis value. A reference line RL3 shows an upper limit of the error specified in the JIS standards (JIS K 2501:2003 potentiometric titration method (acid value)), and a reference line RL4 shows a lower limit of the error specified in the JIS standards. As shown in FIG. 11, errors of respective estimated values fall within the range of errors specified in the JIS standards.

<5-3>

In the above embodiment, the same relationship information is always used to detect the third parameter. However, the relationship information used to detect the third parameter may also be changed according to predetermined conditions. For example, a plurality of pieces of relationship information respectively corresponding to mutually different temperature ranges of the liquid L1 may also be prepared in advance. The plurality of pieces of relationship information may be generated in advance by changing temperatures of a plurality of samples (liquids L1), for example. The plurality of pieces of relationship information may be stored in the storage unit 106.

FIG. 12 is a flowchart showing a procedure for specifying relationship information to be used to detect the third parameter. Processing shown in this flowchart is executed by the control unit 104, for example. As shown in FIG. 12, the control unit 104 detects the temperature of the liquid L1 based on output of the temperature sensor 113 (step S300). The control unit 104 specifies relationship information corresponding to the detected temperature of the liquid L1 (step S310). The third parameter of the liquid L1 may be detected in accordance with the flowchart shown in FIG. 10 with use of the specified relationship information.

The inventor(s) of the present invention found that the relationship between the third parameter and the second parameter may be affected by the temperature of the liquid L1. In this liquid sensor 10, the third parameter is detected based on the temperature of the liquid L1, the relationship information corresponding to the temperature of the liquid L1, and the second parameter. Accordingly, the third parameter is detected with consideration given to the temperature of the liquid L1, and therefore, it is possible to detect the third parameter more precisely with use of this liquid sensor 10.

<5-4>

In the above embodiment, the resistance value R1 is used as the first parameter irrespective of the value of the third parameter. However, the first parameter may also be changed according to the value of the third parameter.

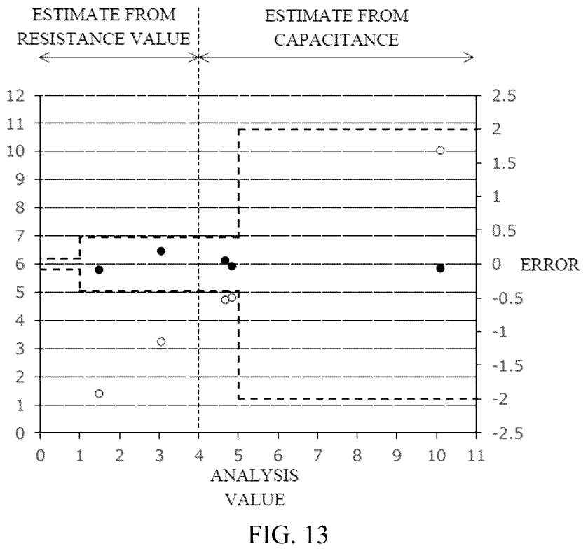

FIG. 13 is a diagram for describing the change of the first parameter. As shown in FIG. 13, for example, the capacitance C1 may be used as the first parameter when the base number (third parameter) is 4 or more, and the resistance value R1 may be used as the first parameter when the base number is less than 4. The inventor(s) found that either of a second parameter calculated based on the resistance value R1 and a second parameter calculated based on the capacitance C1 has a higher degree of correlation with the third parameter varies according to the value of the third parameter. More specifically, the inventor(s) found that the higher the base number is, the higher the degree of correlation between the third parameter and the second parameter calculated based on the capacitance C1 is, and the lower the base number is, the higher the degree of correlation between the third parameter and the second parameter calculated based on the resistance value R1 is.

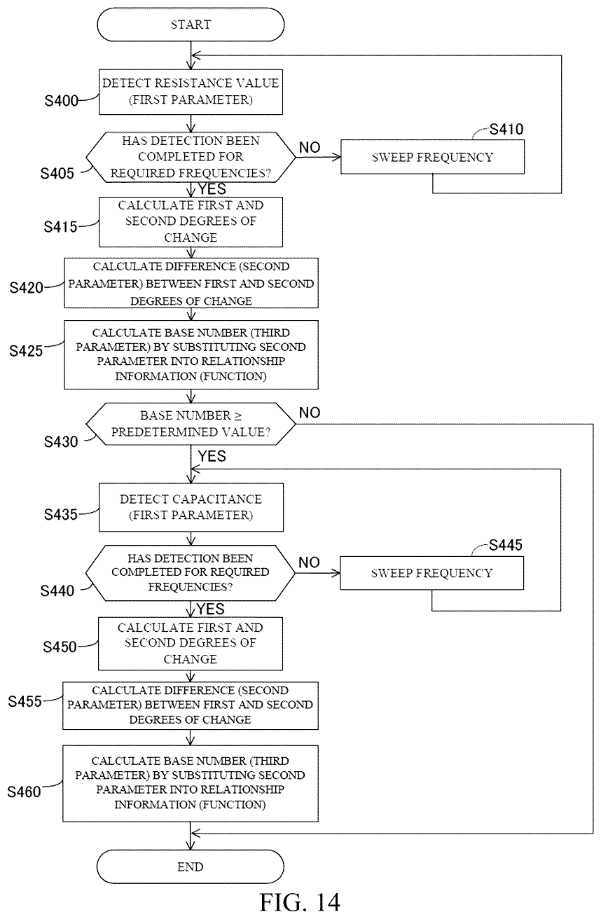

FIG. 14 is a flowchart showing another example of the procedure for detecting the base number of the liquid L1 with use of the liquid sensor 10. Processing shown in this flowchart is executed by the control unit 104.

As shown in FIG. 14, the control unit 104 detects the resistance value R1 (first parameter) of the liquid L1 while a measurement voltage is applied between the pair of electrodes 112 at a predetermined frequency (e.g., any of four frequencies determined in advance in the relationship information) (step S400). The control unit 104 determines whether or not the resistance value R1 has been detected while the measurement voltage is applied between the pair of electrodes 112 at each of a plurality of (e.g., four) predetermined frequencies determined in advance (step S405). Upon determining that the resistance value R1 has not been detected with respect to at least one frequency (NO in step S405), the control unit 104 controls the measurement unit 102 to sweep the frequency of the measurement voltage (step S410). Thereafter, the processing performed in step S400 is performed again.

Upon determining that the resistance value R1 has been detected with respect to all of the predetermined frequencies (YES in step S405), the control unit 104 calculates the first degree of change and the second degree of change based on detection results of the resistance value R1 (step S415). The control unit 104 calculates a difference (second parameter) between the first degree of change and the second degree of change (step S420). The control unit 104 detects the base number (third parameter) of the liquid L1 by substituting the second parameter into relationship information (e.g., a function) stored in the storage unit 106 (step S425). Note that the relationship information used here is generated in advance based on frequency characteristics of the resistance value R1.

The control unit 104 determines whether or not the detected base number is larger than or equal to a predetermined value (e.g., “4”) (step S430). When it is determined that the detected base number is smaller than the predetermined value (NO in step S430), the processing shown in this flowchart ends, and the base number detected in step S425 is taken to be the base number of the liquid L1.

On the other hand, when it is determined that the detected base number is larger than or equal to the predetermined value (YES in step S430), the control unit 104 detects the capacitance C1 (first parameter) of the liquid L1 while the measurement voltage is applied between the pair of electrodes 112 at a predetermined frequency (step S435). The control unit 104 determines whether or not the capacitance C1 has been detected while the measurement voltage is applied between the pair of electrodes 112 at each of a plurality of predetermined frequencies determined in advance (step S440). Upon determining that the capacitance C1 has not been detected with respect to at least one frequency (NO in step S440), the control unit 104 controls the measurement unit 102 to sweep the frequency of the measurement voltage (step S445). Thereafter, the processing performed in step S435 is performed again.

Upon determining that the capacitance C1 has been detected with respect to all of the predetermined frequencies (YES in step S440), the control unit 104 calculates first and second degrees of change based on detection results of the capacitance C1 (step S450). The control unit 104 calculates a difference (second parameter) between the first degree of change and the second degree of change (step S455). The control unit 104 detects the base number (third parameter) of the liquid L1 by substituting the second parameter into relationship information (e.g., a function) stored in the storage unit 106 (step S460). Note that the relationship information used here is generated in advance based on frequency characteristics of the capacitance C1. When the base number is detected in step S460, the processing shown in this flowchart ends, and the base number detected in step S460 is taken to be the base number of the liquid L1.

As described above, in this liquid sensor 10, the first parameter is switched to the resistance value R1 or the capacitance C1 according to the value of the third parameter. Accordingly, a second parameter that has a higher degree of correlation with the third parameter is used to detect the third parameter, and therefore, it is possible to detect the third parameter more precisely with use of this liquid sensor 10.

Example embodiments of the present invention have been described above. That is to say, the detailed description and the appended drawings are disclosed for purposes of illustration. Accordingly, constituent elements described in the detailed description or shown in the appended drawings may include constituent elements that are not essential to solve the problem. Accordingly, even if such non-essential constituent elements are described in the detailed description or shown in the appended drawings, those non-essential constituent elements should not be immediately deemed to be essential.

Also, the above embodiments are merely examples of the present invention in all aspects. Various modifications and alterations can be made on the above embodiments within the scope of the present invention. For example, at least one configuration of any of the embodiments may be combined with at least one configuration of the other embodiments. That is to say, specific configurations may be adopted as appropriate according to the manner of implementation of the present invention.

### LIST OF REFERENCE NUMERALS

10 Liquid sensor
20 Tank
100 Detection system
102 Measurement unit
104 Control unit
106 Storage unit
108 Notification unit
110 Substrate
111 Substrate body
112 Electrode
113 Temperature sensor
DC1, DC2 Degree of change
L1 Liquid
P1 to P5 Point
RL1 to RL4 Reference line

What is claimed is:

1. A liquid sensor comprising:
a pair of electrodes that are used while being immersed in a liquid; and
a control unit configured to sweep a frequency of an AC voltage applied between the pair of electrodes,
wherein the control unit detects an impedance of the liquid for each frequency, and calculates a second parameter relating to a first parameter that is a value correlating with the impedance or the impedance itself,
the second parameter is a difference between a first degree of change and a second degree of change,
the first degree of change and the second degree of change each indicate an amount of change in the first parameter relative to an amount of change in the frequency,
the liquid sensor further comprises a storage unit storing therein relationship information indicating a correspondence between the second parameter and a third parameter correlating with a degradation level of the liquid, and
the control unit detects the third parameter based on the second parameter and the relationship information.

2. The liquid sensor according to claim 1,
wherein the first parameter is a resistance value of the liquid or a capacitance of the liquid.

3. The liquid sensor according to claim 1,
wherein the third parameter is a base number of the liquid or an acid value of the liquid.

4. The liquid sensor according to claim 1, further comprising:
a temperature sensor configured to detect a temperature of the liquid,
wherein the relationship information is set for each temperature of the liquid, and
the control unit detects the third parameter based on the temperature of the liquid, the relationship information corresponding to the temperature of the liquid, and the second parameter.

5. The liquid sensor according to claim 2,
wherein the control unit switches the first parameter to the resistance value or the capacitance according to a detected value of the third parameter.

6. A detection method comprising:
a step of sweeping a frequency of an AC voltage applied between a pair of electrodes immersed in a liquid;
a step of detecting an impedance of the liquid for each frequency; and
a step of calculating a second parameter relating to a first parameter that is a value correlating with the impedance or the impedance itself,
wherein the second parameter is a difference between a first degree of change and a second degree of change,
the first degree of change and the second degree of change each indicate an amount of change in the first parameter relative to an amount of change in the frequency, and
the method further comprises:
a step of storing relationship information indicating a correspondence between the second parameter and a third parameter correlating with a degradation level of the liquid, and
a step of detecting the third parameter based on the second parameter and the relationship information.

7. The liquid sensor according to claim 2,
wherein the third parameter is a base number of the liquid or an acid value of the liquid.

8. The liquid sensor according to claim 2, further comprising:
a temperature sensor configured to detect a temperature of the liquid,
wherein the relationship information is set for each temperature of the liquid, and
the control unit detects the third parameter based on the temperature of the liquid, the relationship information corresponding to the temperature of the liquid, and the second parameter.
